(19) United States
(12) Patent Application Publication
(10) Pub. No.: US 2019/0142979 A1
(43) Pub. Date: May 16, 2019

(54) CONTRAST MEDIUM AND METHODS INVOLVED IN PREPARATION FOR THE DETERMINATION OF PATENCY OF CONDUITS

(71) Applicant: VENKATA NARAYANA RAO CHEGONDI, Palakollu (IN)

(72) Inventor: VENKATA NARAYANA RAO CHEGONDI, Palakollu (IN)

(21) Appl. No.: 16/315,658

(22) PCT Filed: Jul. 7, 2017
(86) PCT No.: PCT/IB2017/054093

§ 371 Date: Jan. 7, 2019

(30) Foreign Application Priority Data
Jul. 8, 2016 (IN) 201641023512

(51) Int. Cl. A61K 49/22; A61K 31/167; A61K 47/02

(52) U.S. Cl. CPC A61K 49/226; A61K 31/167; A61K 49/223; A61K 2123/00; A61K 47/02

(57) ABSTRACT

Exemplary embodiments of the present disclosure are directed towards a physiologically acceptable visualisable contrast medium for the diagnosis of patency of conduits and method of its preparation comprising of an active ingredient comprising of a local anesthetic, whereby the active ingredient incorporated is 2% weight by weight of lidocaine hydrochloride; a sterile mixture comprising of sodium chloride and water, whereby the strength of the sodium chloride incorporated is 0.9% weight by volume in a solution of water.

### TECHNICAL FIELD

The present disclosure generally relates to the field of gel based sonography. More particularly, the present disclosure relates to a contrast medium for the determination of patency of conduits.

### BACKGROUND

Hysterosalpingogram is being performed for the detection of tubular patency more so in Fallopian tubes. The method is performed to check whether the fallopian tubes are open or blocked and the position of the blockage. Some of the known procedures for detection of patency of conduits include;

X-ray HSG (Hystero salpingography) though it is accurate in assessing the tubal patency—it is associated with pain, and the contrast using in this procedure may cause dye allergy and problem of radiation.

Laproscopic chromopertubation: It is also most accurate tool. But it is a surgical procedure—needs anesthesia, the patient has to stay in hospital and most importantly it is very expensive.

Ultrasound guided procedures—Saline Infusion Sonography (SIS) with Color Doppler—using saline as Ultrasound contrast. This is a painful procedure, though less expensive and accurate. The disadvantage is that the tubes cannot be seen. Moreover one only gets indirect evidence of patency and is mainly operator dependent.

HYCOSY (Hysterosalpingo Contrast sonography) done with 3D Ultrasound. It is more accurate. In this, a contrast medium is used, called Echovist. In this procedure, tubes can be visualized clearly and site of block can be assessed. This procedure hits on the economical aspect and is unaffordable for many.

HYFOSY (Hysterosalpingo Foam Sonography) Ultrasound technic using commercially available contrast (Exemfoam gel). Though the procedure is fairly accurate but it is not available across all the corners of the world and is very expensive.

In the light of aforementioned discussion there exists a need for a technique involving a medium whose insertion into the conduits is safe, less or almost no pain, economical and accurate in comparison to the techniques existing in the prior art.

### BRIEF SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary embodiments of the present disclosure are directed towards contrast medium and methods involved in preparation for the determination of patency of conduits

Another exemplary objective of the present subject matter is directed towards a contrast medium safe for a patient upon administration.

Another exemplary objective of the present subject matter is directed towards a contrast medium which is economically viable.

Another exemplary objective of the present subject matter is directed towards a contrast medium which is documentable.

Yet another exemplary objective of the present subject matter is directed towards a contrast medium with viscosity enabling the medium to flow into the conduits easily.

Another exemplary embodiment of the present subject matter is directed towards an active ingredient comprising of a local anesthetic, whereby the active ingredient incorporated is 2% weight by weight of lidocaine hydrochloride.

Yet another exemplary embodiment of the present subject matter is directed towards a sterile mixture comprising of sodium chloride and water, whereby the strength of the sodium chloride incorporated is 0.9% weight by volume in a solution of water.

### BRIEF DESCRIPTION OF DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein:

FIG. 1 is a flow diagram representing a process of preparation of a contrast medium for determination of patency of the conduits, according to an exemplary embodiment of the present disclosure.

FIG. 2 is a flow diagram representing a method of insertion of the contrast medium into a uterine cavity of the patient, according to an exemplary embodiment of the present disclosure.

FIG. 3(A-C) is a pictographic representation of diagnostic images of the fallopian tubes post administration of the contrast medium, according to an exemplary embodiment of the present disclosure.

### DETAILED DESCRIPTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

In accordance to an exemplary embodiment of the present disclosure, the contrast medium for determination of patency of the conduits comprises mainly two components. The first component being not limited to Lidocaine 2% w/w jelly. Lidocaine may also be referred to as Lignocaine and/or Xylocaine and/or lignocaine. Lidocaine is used as a local anesthetic i.e to numb the tissue in a specific area. Lignocaine is available in various forms comprising of Intravenous infusion, Intraosseous infusion, topical ointment, jelly and the like without limiting the scope of the disclosure.

An exemplary component comprising of lidocaine jelly enabled to be utilized as a composition for the contrast medium is a stable aqueous product comprising of lidocaine HCL further comprising of methyl paraben and propyl paraben as preservatives, hypermellose as an emulsifier, thickening and suspending agent which is enabled to be used as a substitute to animal gelatin and sodium HCL in order to adjust the ph between 6-7. This ensure the neutrality of the product as too much acidity or too much basicity may harm the patient as the medium is intended to be inserted into the vagina of the patient undergoing the determination of patency.

Lidocaine is considered to be highly safe upon injection into uterine cavity or into peritoneal cavity. Alteration of fertility levels is also not reported post administration.

The second component incorporated in the contrast medium is a normal saline solution. Normal saline solution comprises of Sodium chloride and water and is a sterile mixture. It is 0.9% strength of sodium chloride (salt) solution in water. The sterility of natural saline makes it a safe product for administration.

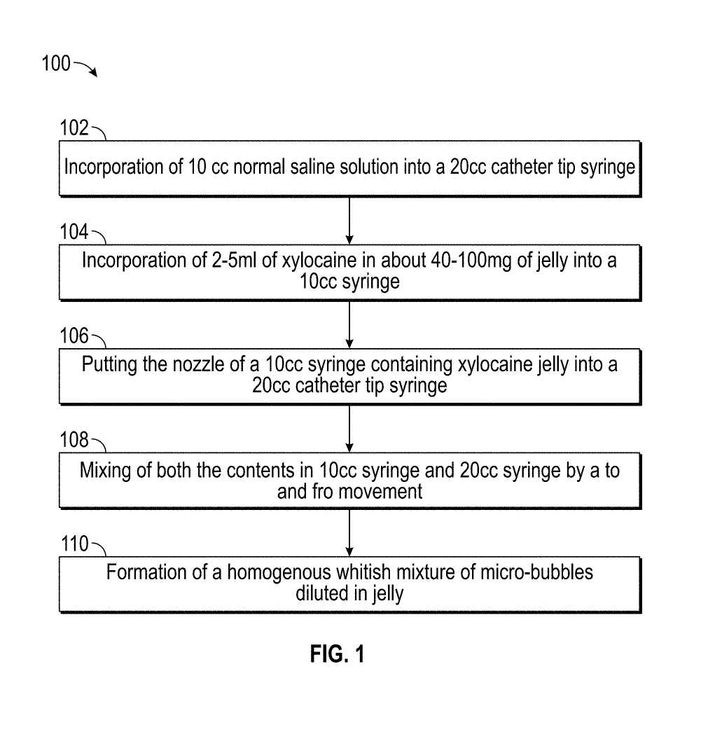

Referring to FIG. 1 is a flow diagram 100, representing a process of preparation of a contrast medium for determination of patency of the conduits, according to an exemplary embodiment of the present disclosure. The process commences at step 102 by the incorporation through suction of 10 cc normal saline solution into a 20 cc catheter tip syringe. At step 104, 2-5 ml of Xylocaine and/or lignocaine in about 40-100 mg of jelly is incorporated into a 10 cc syringe. Further, the nozzle of a 10 cc syringe containing Xylocaine and/or lignocaine Jelly is put into a 20 cc catheter tip syringe at step 106. Mixing of both the contents in 10 cc syringe and 20 cc syringe by a to and from movement takes place at step 108. This leads to formation of a homogenous whitish mixture of micro-bubbles diluted in jelly at step 110.

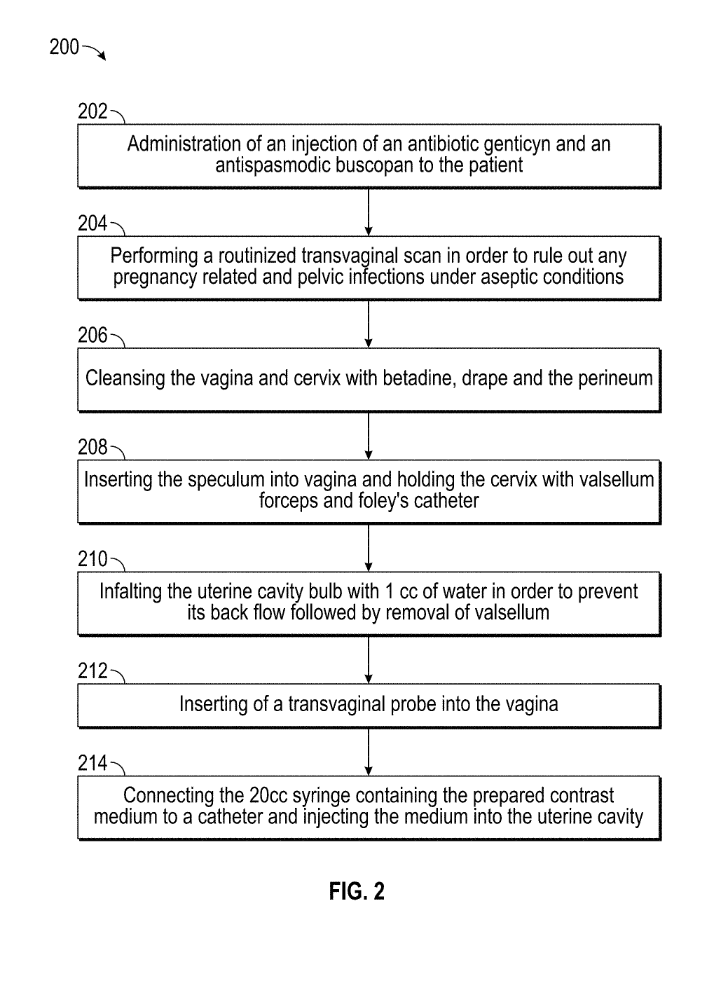

Referring to FIG. 2 is a flow diagram 200, representing a method of insertion of the contrast medium into a uterine cavity of the patient, according to an exemplary embodiment of the present disclosure. The method starts at step 202 with the administration of an injection containing an antibiotic Genticyn and an antispasmodic Buscopan to the patient. Performing a routinized transvaginal scan in order to rule out any pregnancy related and pelvic related infections under aseptic conditions is done at step 204. A transvaginal ultrasound is a type of pelvic ultrasound used by doctors to examine female reproductive organs. This includes the uterus, fallopian tubes, ovaries, cervix, and vagina.

Cleansing of the vagina and cervix is done with Betadine, Drape and the Perineum at step 206. Insertion of speculum into Vagina and holding the cervix with valsellum forceps and Foley's catheter is done at step 208. Inflation of the uterine cavity bulb with 1 cc of water is done in order to prevent its back flow followed by removal of valsellum at step 210. Insertion of a transvaginal probe into the vagina at step 212 and finally, at step 214 connecting the 20 cc syringe containing the prepared contrast medium to a catheter is done followed by injecting the medium into the uterine cavity.

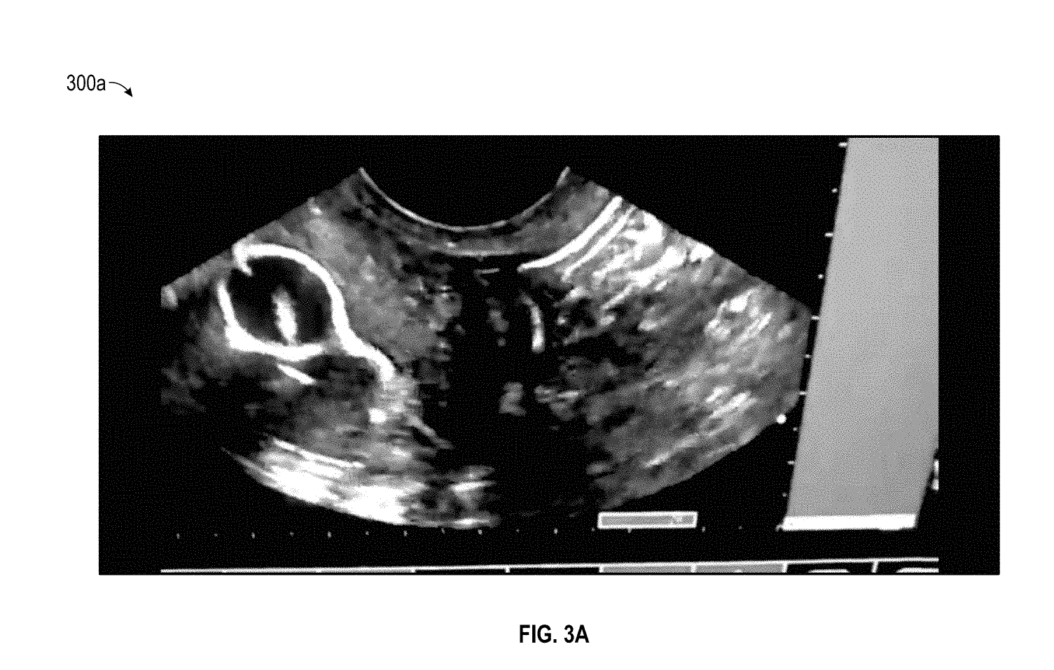

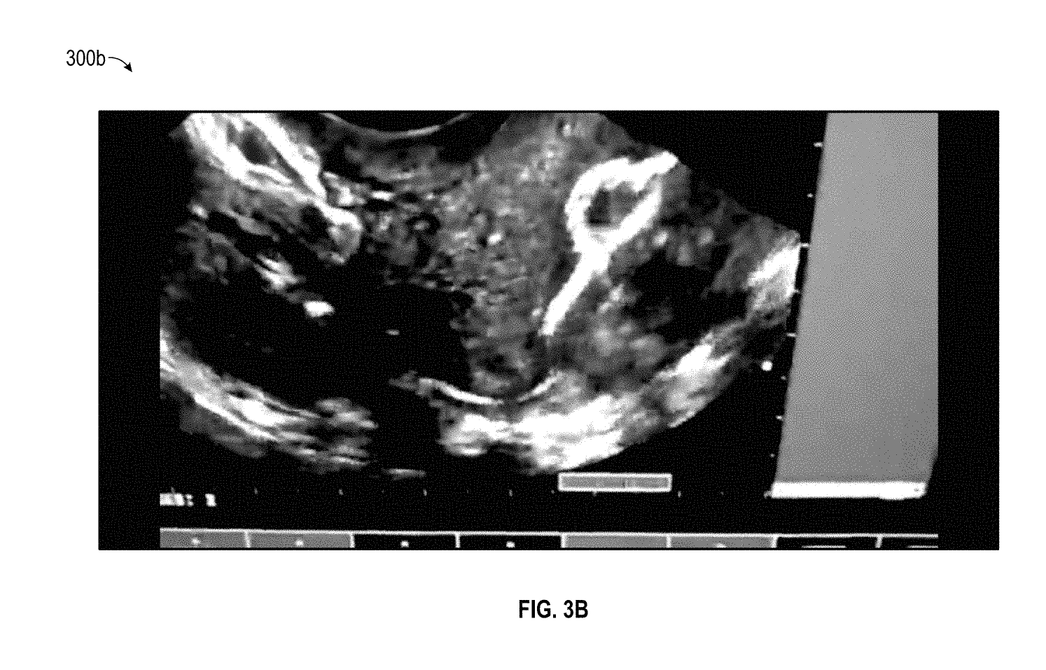

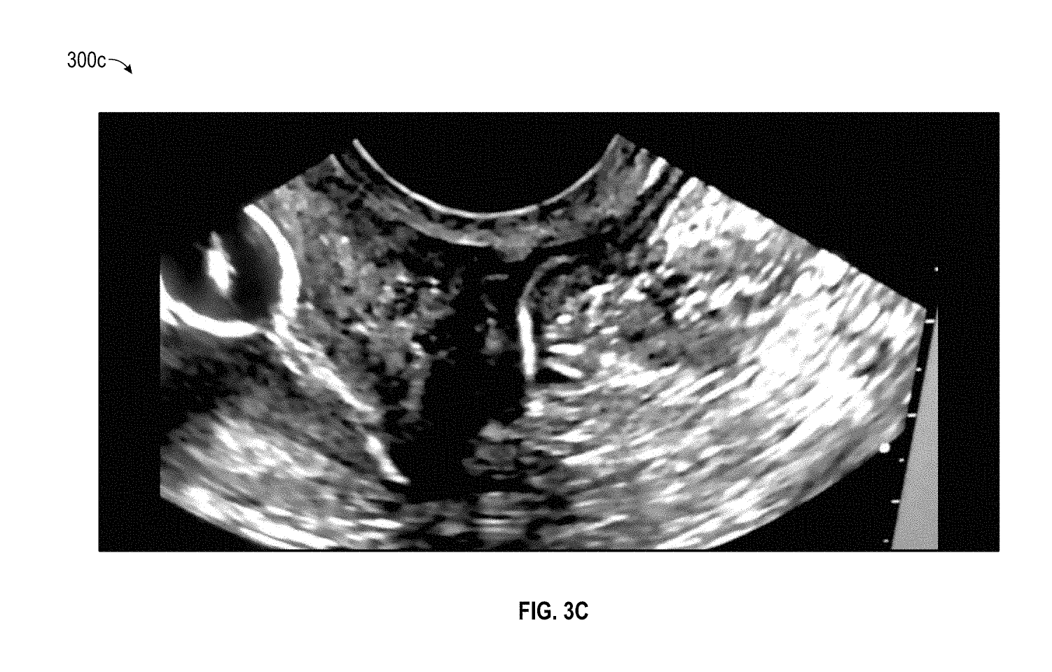

Referring to FIG. 3(A-C) is a pictographic representation 300(a-c), of diagnostic images of the fallopian tubes post administration of the contrast medium, according to an exemplary embodiment of the present disclosure. Technically, the test it not configured to have images of both the fallopian tubes at the same time. Each time the analysis of one side is a possibility. The medium is enabled to be visualized upon

The pictographic representation of FIG. 3A depicts the diagnostic image 300a using the contrast medium wherein, the entire fallopian tubes are seen along with the spillage of the contrast medium (represented as the illuminating passage) thus concluding the patency of the fallopian tubes.

The pictographic representation of FIG. 3B depicts the diagnostic image 300b using the contrast medium wherein, the spillage of the contrast medium not seen and only a part of the fallopian tube is visible thus a distal blockage of the fallopian tube may be speculated.

The pictographic representation of FIG. 3C depicts the diagnostic image 300c using the contrast medium wherein, the entire fallopian tube is visible and spillage of the contrast medium is also seen thus concluding the patency of the fallopian tubes.

Below table shows study of 5 patients who underwent x-ray HSG, Xylocaine and/or lignocaine in natural saline medium and Laproscopic Chromopertubation.

| | | | Xylocaine | |
|---|---|---|---|---|
| | | | and/or | |
| | | | lignocaine | |
| | | | in natural | |
| | Fallopian | | saline | Laproscopic |
| Case | Tubes | HSG | medium | chromoperturbation |
| 1 | Both | Patent | Patent | Patent |
| | tubes | | | |
| 2 | Both | Failed | Patent | Patent |
| | tubes | | | |
| 3 | Left | Blocked | Patent | Patent |
| | Right | Patent | Patent | Patent |
| 4 | Right | Blocked | Blocked | Blocked |
| | Left | Blocked | Blocked | Blocked |
| 5 | Right | Blocked | Patent | Patent |
| | Left | Patent | Patent | Patent |

Although the present disclosure has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub combinations of the various features described herein above as well as variations and modifications thereof, which would occur to persons skilled in the art upon reading the foregoing description.

What is claimed is:

1. A physiologically acceptable visualisable contrast medium for the diagnosis of patency of conduits comprising of;
an active ingredient comprising of a local anesthetic, whereby the active ingredient incorporated is 2% weight by weight of lidocaine hydrochloride;
a sterile mixture comprising of sodium chloride and water, whereby the strength of the sodium chloride incorporated is 0.9% weight by volume in a solution of water.

2. The visualisable contrast medium of claim 1, enabled to penetrate conduits of at least one of human and animal.

3. Preparation of a physiologically acceptable visualisable contrast medium for the diagnosis of patency of conduits, method comprising of;
Incorporating a 10 cc of a sterile mixture comprising of sodium chloride and water into a 20 cc catheter tip syringe;
Incorporating at least 2 ml ranging up to 5 ml of lidocaine hydrochloride in at least 40 mg ranging up to 100 mg of jelly in a 10 cc syringe;
Mixing of both the contents in 10 cc syringe and 20 cc syringe by a to and from movement; whereby the mixing leads to formation of a homogenous whitish mixture of micro-bubbles diluted in jelly.

4. A visualisation procedure of providing physiologically acceptable visualisable contrast medium for the diagnosis of patency of conduits comprising of;
Inserting a speculum into Vagina and holding the cervix with valsellum forceps and Foley's catheter;
Inflating a uterine cavity bulb with 1 cc of water whereby, the backflow of the physiologically acceptable visualisable contrast medium is controlled after the removal of the valsellum;
Inserting a transvaginal probe into the vagina;
Connecting the 20 cc syringe containing the prepared physiologically acceptable visualisable contrast medium to a catheter and injecting the medium into the conduit for visualization of patency of the conduits.

5. The method of claim 4, wherein the conduit may be at least one of fallopian tubes, vascular conduits, uterine cavity and ureters.

6. The method of claim 4, wherein the visualization procedure is at least one of Hystero gel sonosalpinograhy, sonography, ultrasound, fluoroscopy, laparoscopic chromoperturbation, nuclear medicine techniques, magnetic resonance techniques, Hystero salpingo contrast sonography and Hystero salpingo foam sonography.

7. The method of claim 4, wherein the physiologically acceptable visualisable contrast medium is subjected to at least one conduit for visualization procedure in a predetermined quantity.

8. The method of claim 4, wherein the physiologically acceptable visualisable contrast medium is allowed to remain in at least one of the conduits for a predetermined amount of time.
